(19) United States
(12) Patent Application Publication
(10) Pub. No.: US 2004/0254619 A1
(43) Pub. Date: Dec. 16, 2004

(54) Apparatus and method for photothermal and photochemical medical treatments with incoherent light

(76) Inventors: Daniel Feuermann, Ben-Gurion (IL); Jeffrey M. Gordon, Ben-Gurion (IL); Harald Ries, Muenchen (DE); Kim Choon Ng, Singapore (SG); Hui Tong Chua, Singapore (SG); Michael Altura, Los Angeles, CA (US)

Correspondence Address: LACKENBACH SIEGEL, LLP, LACKENBACH SIEGEL BUILDING, 1 CHASE ROAD, SCARSDALE, NY, 10583, US

(21) Appl. No.: 10/770,689

(22) Filed: Feb. 2, 2004

Related U.S. Application Data
(60) Provisional application No. 60/478,759, filed on Jun. 16, 2003.

(51) Int. Cl. A61N 5/06

(52) U.S. Cl. 607/88

(57) ABSTRACT

An apparatus and method for non-imaging optical systems to couple incoherent light from a source such as high-brightness lamps into optical fibers, and to deliver the light into the human body for photothermal and/or photochemical surgery. The incoherent light radiated from the light source is concentrated with a non-imaging light concentrator and the light is delivered to a body tissue for photothermal and/or photochemical medical treatment. The concentrator captures nearly the light source's full radiative output, and concentrates the collected radiation back to power densities close to the power densities of the hot plasma regions at the core of the light source. The concentrator couples the light into a photonic conduit such as optical fibers, and the photonic conduit delivers the concentrated intense light to surgical applications including contact (interstitial) procedures and non-contact treatments (within the internal body cavities as well as the surfaces of organs), and inside the body. The light radiated from the light source may be spectrally filtered, in particular for procedures that benefit from a narrow wavelength spectral window. The apparatus and method are compact, highly efficient, portable, and relatively inexpensive.

### FIELD OF THE INVENTION

The invention generally relates to non-imaging optical systems to couple incoherent light into optical fibers, and more particularly to delivering light for photothermal and photodynamic medical treatments.

### BACKGROUND

Many laser medical procedures involve coupling laser light into optical fibers that serve as photonic conduits to the treated area, be it interstitial (light being injected to the interior region of an organ in a contact procedure) or irradiation of tissue surfaces (non-contact treatments). Photothermal procedures involve killing tissue by rapid and highly localized heating to at least 50° C. In contrast, photochemical procedures—most notably photodynamic therapy—kill cancerous tissue with photonically-activated chemical reactions based on the selective uptake of photosensitizing agents by malignant cells. Laser fiber-optic surgery can be minimally invasive, often performed under local anesthesia in outpatient clinics, with associated benefits that include a markedly reduced risk of infection, far less pain to the patient, and faster recovery.

Most photothermal treatments exploit solely the immense power density attainable with lasers, rather than laser coherence or monochromaticity. In killing sizable (i.e., thick) tumors, the optical and thermal penetration required dictate visible or near-infrared wavelengths. Excessive healthy tissue must not be destroyed and the exposure must be completed within a few minutes, with the ability to kill tumors of the order of one to several cm³.

This, in turn, places stringent demands on both the delivered power and power density. In photothermal surgery, a minimum of several Watts is typically required at power densities of at least several W/mm². In contrast, photodynamic therapy requires of the order of 10⁻³ to 10⁻² W/mm², but all within a wavelength window of no more than a few tens of nanometers.

The key drawback of surgical lasers, and laser fiber-optic surgery in general, is exorbitant price, compounded by their size and the required infrastructure, which militate against portability. A potentially inexpensive, practical and more portable alternative is to use incoherent light from conventional compact lamps. But until quite recently, conventional lamps have been intrinsically disqualified due to inadequate brightness.

A ultra-high-brightness short-arc plasma discharge lamp as shown in FIG. 1 and discussed in “Technology and applications: XBO theatre lamps”, Technical brochure, Osram GmbH, Photo-optic Division, Nonnendammalle 44-61, D-13625, Berlin, Germany (Osram 2002). FIG.1 shows a schematic of a short-arc Xenon plasma discharge lamp 10. The lamp 10 includes a cathode base 12 connected to a cathode 18 configured with a lamp shaft 14 to provide the cathode 18 within the lamp bulb 16. The lamp bulb 16 has an exhaust tube 22 and an ignition wire 20 within the lamp bulb, together with an anode 24. The anode is connected to the electrode rod 26 to an anode base 30. A seal 28 is formed around the electrode rod 26.

This technology appears to offer stable plasma emissions, lamp lifetimes of the order of hundreds of hours, a spectral distribution similar to that of the sun, i.e., from the violet through the near-infrared, as shown in the graph of FIG. 2, and a spectral power density at the source significantly greater than what is required in many photothermal surgical procedures. FIG.2 shows a graph of the spectral irradiance of a typical commercially available ultra-bright short-arc Xenon plasma discharge lamp, for example as shown in FIG. 1. The graph of FIG.2 shows the visible region 32 of light for a Xenon plasma discharge lamp along curve 34 and a black body radiator along curve 36. These properties imply that if one could concentrate light emitted by the lamp to power densities near those of the hot plasma source and couple the light into high-transmissivity optical fibers, a potentially inexpensive and practical alternative to many laser fiber-optic surgical systems is provided.

Patent applications have been filed for devices that incorporate (a) concentrating the light from bright plasma discharge lamps, (b) coupling the light into optical fibers and (c) using the output for an assortment of medical treatments, for example U.S. Pat. No. 4,860,172 issued 22 Aug. 1989 to Sclager et al., International Patent Publication No. WO 93/00,551 published 7 Jan. 1993 in the name of Ghaffari, U.S. Pat. No. 5,707,401 issued 13 Jan. 1998 to Talmore, and International Patent Publication No. WO 00/77,446 published 21 Dec. 2000 in the name of Tissuemed Limited.

The earlier attempts deploy an ellipsoidal reflector as a concentrator of lamplight, or as the primary stage of a dual- or multi-stage optical concentrator. Such imaging strategies are inherently flawed, in that the strategies (1) fail to collect most of the emitted light, and (2) dilute the attainable power density. The supplementary second- or higher-stage concentrators in the cited previous devices neither offer a significant boost in flux concentration nor enhance collection efficiency. The result is an optical system that delivers a small fraction of the lamp's utilizable output, at power densities that fall short of the high values required in photothermal surgery. The power densities are below the threshold values within the narrow wavelength windows for most photochemical treatments. The lower brightness of earlier generations of arc discharge lamps exacerbated the problem; although that problem appears to have been mitigated in the latest generations of Xenon short-arc lamps as discussed in Osram 2002.

The fact that ultra-bright incoherent light can indeed generate the type, extent and rate of photothermal tissue transformations (cell death) previously deemed achievable only with surgical lasers was recently established experimentally with highly concentrated sunlight in compact and potentially inexpensive devices. The ephemeral nature of solar radiation severely restricts the feasibility of solar surgery.

Therefore, there is a need for an apparatus and method for use of incoherent light in medical treatments such as photothermal and/or photochemical medical treatments.

### SUMMARY

An aspect of the invention provides an apparatus for photothermal and/or photochemical medical treatments. The apparatus comprises a light source for radiating high intensity incoherent light; a non-imaging light concentrator for concentrating the radiated light from the light source; and a photonic conduit for delivering the light to a body tissue at a location remote from the light source for photothermal and/or photochemical medical treatment.

An embodiment provides the photonic conduit comprising fiber optics. The non-imaging light concentrator may comprise at least one concentrator unit, each concentrator unit for concentrating the radiated light. The concentrator may have an optical coupler for the light to pass through to accommodate any mismatch in the numerical aperture of the light. The light source may comprise a short-arc plasma discharge lamp.

Another aspect of the invention provides a method for photothermal and/or photochemical medical treatments. The method comprises radiating high intensity incoherent light from a light source; concentrating the radiated light from the light source with a non-imaging light concentrator; and delivering the light in a photonic conduit to a body tissue at a location remote from the light source for photothermal and/or photochemical medical treatment.

An embodiment provides delivering light through the photonic conduit comprising fiber optics. Concentrating the radiated light may comprise at least one concentrator unit, each concentrator unit for concentrating the radiated light. The method may further comprise an optical coupler for the light to pass through to accommodate any mismatch in the numerical aperture of the light. The radiating high intensity incoherent light may be radiated from a short-arc plasma discharge lamp light source.

### BRIEF DESCRIPTION OF THE DRAWINGS

These and other features, objects and advantages of embodiments of the invention will be better understood and readily apparent to one of ordinary skill in the art from the following description, in conjunction with the drawings, in which:

FIG. 1 shows a schematic diagram of a conventional short-arc Xenon plasma discharge lamp;

FIG. 2 shows a graph of the spectral radiant intensity versus wavelength of a conventional Xenon plasma discharge lamp and a black body radiator;

FIG. 3A-C show examples of a concentrator unit for an optical device in accordance with an embodiment of the invention;

FIG. 4 shows an optical system in accordance with an embodiment of the invention;

FIG. 5 shows an optical fiber output in accordance with an embodiment of the invention; and

FIG. 6A-B shows optical fiber outputs taken along line B-B′ of FIG. 5 in accordance with an embodiment of the invention.

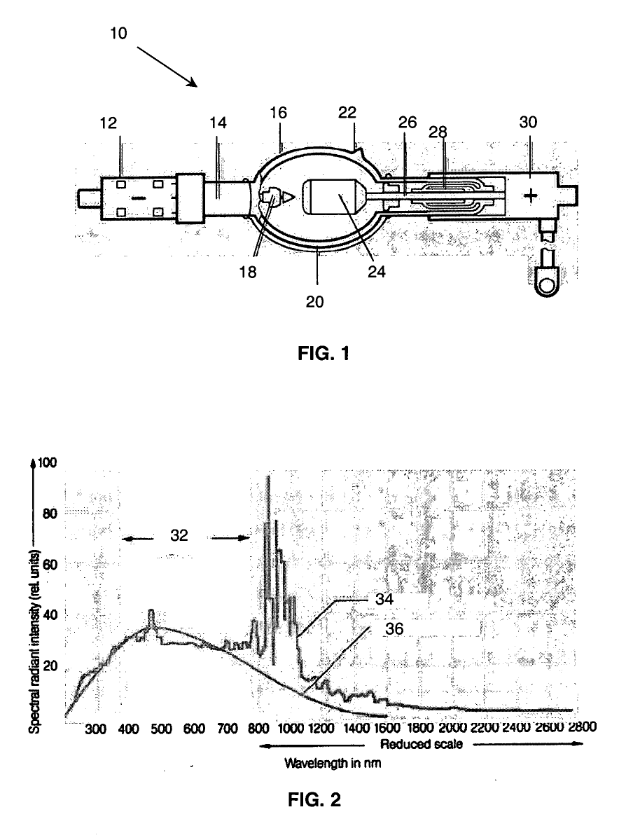

### DETAILED DESCRIPTION

If radiative transfer rather than image fidelity is vital, non-imaging optics offer designs that are markedly superior to those of conventional imaging optics in terms of collection efficiency and achievable flux concentration. Many non-imaging systems can approach the fundamental or so-called thermodynamic limit to optical concentration, which is typically significantly greater than the flux concentration and collection efficiency of imaging designs such as elliptical reflectors and spherical lenses. These non-imaging systems can comprise contoured reflectors (mirrors), aspheric lenses, or lens-reflector combinations (including concentrators based on imaging designs—that is concentrators tailored so that the one-to-one correspondence between a ray from the source to the ray at the target preserves image fidelity to the maximum extent possible), where each contoured surface (mirror or lens) is tailored for maximum optical throughput at maximum concentration.

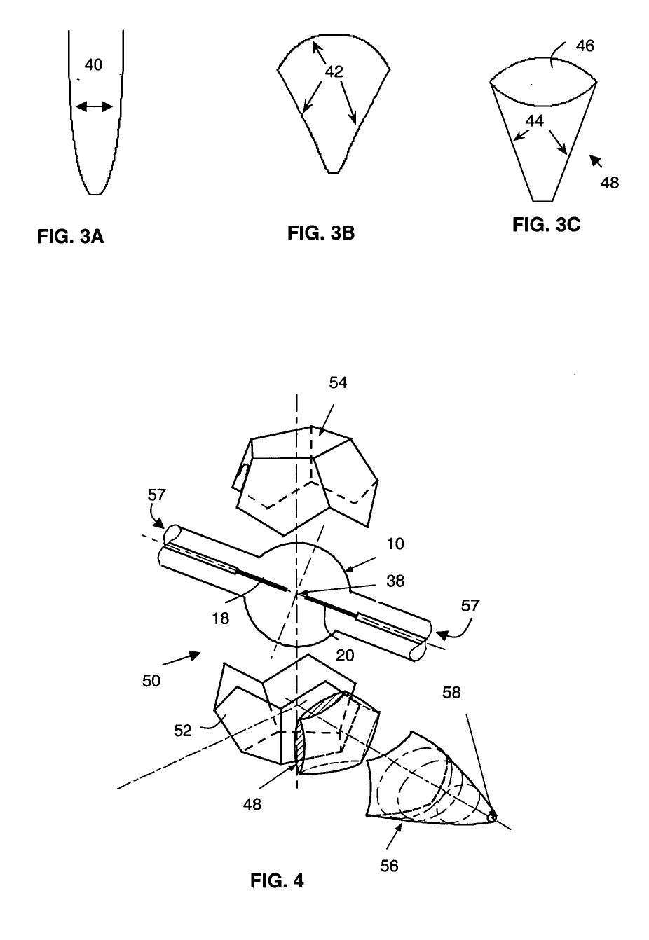

FIGS. 3A-3C show examples of the individual concentrator units, several or many of which comprise the proposed optical device for concentrating lamp radiative emissions. The ensemble of individual concentrator units may also be constructed as one grand concentrator unit where the fusing can be achieved for example by plastic molding technique. A pure reflective (contoured) mirror 40 is shown in FIG. 3A, and a pure refractive aspheric contoured lens 42 with total internal reflection is shown in FIG. 3B. A lens-mirror combination 48 of a contoured mirror 44 and lens 46 is shown in FIG. 3C.

An embodiment of the invention incorporates non-imaging devices of these sorts, which surround an ultra-high-brightness lamp, to efficiently collect emitted radiation, concentrate the emitted radiation to the highest flux concentration commensurate with system requirements, and couple the light into optical fibers, of up to several meters in length for example, for use in surgical applications. FIG. 4 provides one such example, as shown in U.S. Pat. No. 6,336,738 issued on 8 Jan. 2002 to Feuermann, et al., incorporated herein by reference, for use in other applications. This embodiment shown in FIG. 4 includes a 12-sided enclosure, and concentrators comprised of a lens-mirror combination.

FIG. 4 shows an example of a maximum-performance optical system 50 for collecting nearly all of a lamp's light output and concentrating the light output to a flux level close to that of the radiating region 38 of the light source 10. The example shows a lens-mirror combination comprising a lens 48 and a reflector 56, of which one embodiment is shown in FIG. 3C, for each of the concentrator units. Two concentrator units are omitted in the vicinity of the two electrodes 18, 20, which allows easy access for a lamp replacement as well as forced-air cooling. The system 50 further comprises an enclosure 54 with concentrator entrance apertures 52, and a reflector 56 having an exit 58 to an optical fiber tip forming a photonic conduit.

The exit numerical aperture (NA) of the concentrator should not exceed that of the optical fibers (otherwise the light leaks through the fiber cladding and does not reach the surgical target). Attainable flux concentration is proportional to the square of the NA at the exit of the optical system. Optical fibers with relatively high NA and high transmissivity over the full visible and near-infrared are commercially available. When only restricted wavelength windows are needed, even higher values of transmissivity can be realized with a variety of optical fibers that have already been developed commercially for laser surgical applications.

In addition to the system disclosed in U.S. Pat. No. 6,336,738, the device herein may more generally comprise the following features:

(a) An enclosure with an arbitrary number of concentrators is allowed. The configuration of FIG. 4 is designed for maximum collection with a minimum number of identical concentrator units. By allowing a small sacrifice in collection efficiency—for example, permitting 10-20% of the emitted light to fall on non-collecting areas among the concentrators—and by further not requiring the concentrator units to be identical, any number from a few to tens of concentrator units can be introduced. Note that the individual concentrator units may also be achieved for example by plastic molding technique.

(b) Each non-imaging concentrator may comprise a mirror (pure reflective), or an aspheric lens (pure refractive), or a lens-mirror combination.

(c) Each concentrator exit may be filled with one or more (e.g., a bundle of) optical fibers.

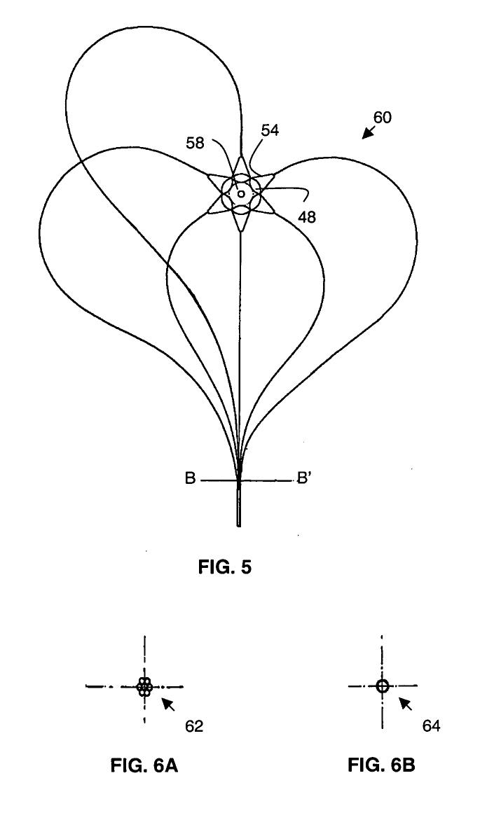

(d) The fibers emerging from the device can either be combined into a closely-packed bundle or fused into one or more fibers, each of which serves as the power delivery channel, as shown for example in FIG. 5. This channel is coupled to the detachable, sterilizable or disposable surgical fiber used in the medical procedure. FIG. 5 shows the fiber optic portion of the photonic conduit. In embodiments the optical fiber output can either be formed (a) into a single bundle 62 as shown in FIG. 6A, or (b) fused into one or more delivery fibers 64 as shown in FIG. 6B, for insertion into the body for photonic surgery, as currently done in laser fiber-optic surgical techniques. This cross-section B-B′ of FIG. 5 is shown as different embodiments in FIG. 6A and 6B, and pertains to a different lamp-concentrator geometry than shown in FIG. 4, although a lens-mirror concentrator unit is retained.

(e) An optical coupler (e.g., a collimator or concentrator) may be inserted between the fiber bundle that emanates from the concentrators and the fiber or fibers that deliver light into the body, toward accommodating a mismatch in their respective NA values.

(f) The concentrators can be distanced from the lamp's quartz envelope to lessen the cooling load on the lamp and optical elements, or to introduce spectral filters (as described below). Increasing the gap between the lamp envelope and the concentrators requires designing for increased flux concentration.

(g) Active cooling of the lamp and optical elements is achieved by forced air ventilation, for example, through the apertures 57 in enclosure 54 around the lamp envelope containing the lamp's electrodes, as shown in FIG. 4.

Continuous control of the radiative power delivered or power control to the surgical target can be achieved in several ways, for example:

(1) Varying the electrical power input to the lamp (although lamp operation characteristics usually restrict this to only a few tens of percent of the nominal operating power).

(2) Controlled movement of the optical fibers relative to their maximum-performance positions in the coupling region between the fibers emanating from the concentrators and those that deliver light to the surgical target, by lateral or longitudinal misalignment.

(3) Introducing a controllable iris in the fiber coupling region.

Spectral filtering or selecting narrow wavelength ranges can be beneficial in some photothermal surgical procedures and is used in photochemical procedures. Spectral filtering can be achieved in one of several fashions, which may for example be achieved by:

(1) Introducing selective band-pass windows between the lamp and the entrance to each concentrator;

(2) Depositing selective band-pass coatings on the lenses, for example, if the units of FIG. 3B or 3C are used;

(3) Depositing selective band-pass coatings on the reflectors, for example, if the units of FIG. 3A or 3C are used;

(4) Exploiting chromatic aberration in the concentrator lenses (for example, when the units of FIG. 3B or 3C are used) toward insuring that most light outside the desired wavelength window is rejected from the optical system before the light reaches the concentrator's exit; and/or

(5) Choosing optical fibers with high core absorption and/or substantial light leakage into the cladding, at wavelengths outside of the prescribed wavelength window.

Realizing maximum-performance fiber-optic remote irradiation systems creates the possibility of realistically providing surgical applications that could not previously be realized with incoherent photonic systems. One important class is interstitial photothermal surgery: killing tumors in internal organs such as the liver, prostate, bladder, pancreas, esophagus, kidney, brain, breast and cervix. The fact that laser surgical effects, i.e., tissue death deriving from coagulation, dehydration, carbonization and/or ablation, can indeed be achieved with incoherent light of immense power density has recently been demonstrated experimentally with sunlight. Photonic and medical processes can be implemented using an ultra-bright source, such as short-arc discharge lamps rather than the surface of the sun. While solar surgery has its appeal for sun-belt regions, solar surgery clearly is severely restricted by solar availability. Consequently lamp-based fiber-optic surgical systems are advantageous.

Photodynamic therapy with incoherent lamps has been limited to superficial irradiation schemes, and even then with marginal capability due to the delivered power density within the typical utilizable wavelength window of no more than a few tens of nanometers being too dilute. In a non-imaging fiber-optic system with ultra-bright incoherent lamps, spectral (chromatic) filtering can be introduced, as delineated above. Materials for the spectral filtering strategies described above are commercially available, especially for devices as small as those needed with current commercial ultra-bright lamps, for which the diameter of the hot utilizable discharge region is of the order of about 0.5-1 mm, and for which the total fiber cross-sectional area is therefore of the order of one to several mm².

Embodiments of the invention allow a broader range of photothermal and/or photochemical medical treatments to be performed with incoherent light than previously recognized. One reason for this involves the power density and power delivery thresholds for effective surgical treatments that until now were only deemed possible with lasers, with the key property of the laser being its immense deliverable power density at significant average power. Only with maximum-performance non-imaging concentrators can the requisite collection efficiencies and delivered power densities be attained.

Another reason relates to the surgical constraint that minimally-invasive photonic surgery inside the body limits the diameter available for the power-delivery fiber(s) to around 2 mm for the surgical incision. Hence even if the internal interstitial tissue surface or tissue-cavity environment can be destroyed with power densities below those that emerge at the distal circular tip of the optical fiber(s), the necessary absolute power must be condensed into a cross-sectional area of the order of one to a few mm² for maximum allowable entry incisions.

It will be appreciated that although specific embodiments of the invention have been described herein for purposes of illustration, various modifications may be made without departing from the scope of the invention as defined by the appended claims.

What is claimed is:

1. An apparatus for photothermal and/or photochemical medical treatments, comprising:
a light source for radiating high intensity incoherent light;
a non-imaging light concentrator for concentrating the radiated light from the light source; and
a photonic conduit for delivering the light to a body tissue at a location remote from the light source for photothermal and/or photochemical medical treatment.

2. An apparatus of claim 1, wherein the light conduit comprises fiber optics.

3. An apparatus of claim 1, wherein the non-imaging light concentrator comprises at least one concentrator unit, each concentrator unit being operable to concentrate the radiated light.

4. An apparatus of claim 3, wherein the at least one concentrator unit comprises at least one pure reflective contoured mirror.

5. An apparatus of claim 3, wherein the at least one concentrator unit comprises at least one pure reflective aspheric lens.

6. An apparatus of claim 3, wherein the at least one concentrator unit comprises at least one lens-mirror combination.

7. An apparatus of claim 6, wherein the at least one concentrator unit comprising at least one lens-mirror combination comprises at least one concentrator based on an imaging design.

8. An apparatus of claim 1, wherein the non-imaging light concentrator is a mirror, an aspheric lens, or a lens-mirror combination.

9. An apparatus of claim 1, wherein the non-imaging light concentrator comprises a concentrator exit for emitting the light.

10. An apparatus of claim 9, further comprising a photonic conduit coupled to the concentrator exit.

11. An apparatus of claim 10, wherein the photonic conduit is a fiber optics means comprising at least one optical fiber.

12. An apparatus of claim 11, wherein the fiber optic means comprises at least two optical fibers that are fused into one optical fiber to form a channel.

13. An apparatus of claim 11, wherein the fiber optic means comprises at least two optical fibers that form a bundle to form a channel.

14. An apparatus of claim 1, further comprising an optical coupler for the light to pass through to accommodate any mismatch in the numerical aperture of the light.

15. An apparatus of claim 1, wherein the concentrator further comprises a spectral filter.

16. An apparatus of claim 15, wherein the spectral filter comprises a selective band-pass coating on the concentrator.

17. An apparatus of claim 11, wherein the concentrator comprises a concentrator lens arrangement having chromatic aberration in the concentrator lenses to ensure that light outside a desired wavelength window is removed from the light before the light reaches the exit.

18. An apparatus of claim 11, wherein the fiber optic means comprises optical fibers having high core absorption and/or substantial light leakage into the cladding, at wavelengths outside of a desired wavelength window.

19. An apparatus of claim 1, wherein the radiative power of the light delivered to the target is controlled by varying the electrical power input to the light source.

20. An apparatus of claim 11, further comprising a fiber coupling region, wherein the fiber optic means comprises at least two optical fibers and the radiative power of the light delivered to the target is controlled by movement of the optical fibers relative to maximum-performance positions of each optical fiber in the coupling region between the fibers emanating from the concentrator and the fibers that deliver light to the surgical target, by lateral or longitudinal misalignment.

21. An apparatus of claim 11, further comprising a fiber coupling region and an iris located therein, wherein the fiber optic means comprises at least two optical fibers and the radiative power of the light delivered to the target is controlled by the iris in the fiber coupling region.

22. An apparatus of claim 1, wherein the light source comprises a short-arc plasma discharge lamp.

23. A method for photothermal and/or photochemical medical treatments, said method comprising the steps of:
radiating high intensity incoherent light from a light source;
concentrating the radiated light from the light source with a non-imaging light concentrator; and
delivering the light in a photonic conduit to a body tissue at a location remote from the light source for photothermal and/or photochemical medical treatment.

24. A method of claim 23, wherein the delivering light conduit comprises fiber optics.

25. A method of claim 23, wherein concentrating the radiated light comprises concentrating the radiated light with at least one concentrator unit.

26. A method of claim 25, wherein concentrating the radiated light comprises concentrating the radiated light with at least one pure reflective contoured mirror.

27. A method of claim 25, wherein concentrating the radiated light comprises concentrating the radiated light with at least one pure reflective aspheric lens.

28. A method of claim 25, wherein concentrating the radiated light comprises concentrating the radiated light with at least one lens-mirror combination.

29. A method of claim 28, wherein concentrating the radiated light with at least one lens-mirror combination comprises concentrating the radiated light with at least one concentrator based on an imaging design.

30. A method of claim 23, wherein concentrating the radiated light, the non-imaging light concentrator is a mirror, an aspheric lens, or a lens-mirror combination.

31. A method of claim 23, wherein concentrating the radiated light comprises the non-imaging light concentrator comprising a concentrator exit for emitting the light.

32. A method of claim 31, further comprising a photonic conduit coupled to the concentrator exit.

33. A method of claim 32, wherein the photonic conduit is a fiber optics means comprising at least one optical fiber.

34. A method of claim 33, wherein delivering the light the fiber optic means comprises at least two optical fibers that are fused into one optical fiber to form a channel.

35. A method of claim 33, wherein delivering the light the fiber optic means comprises at least two optical fibers that form a bundle to form a channel.

36. A method of claim 23, further comprising an optical coupler for the light to pass through to accommodate any mismatch in the numerical aperture of the light.

37. A method of claim 23, wherein the concentrator further comprises a spectral filter.

38. A method of claim 33, wherein the concentrator comprises a concentrator lens arrangement having chromatic aberration in the concentrator lenses to ensure that light outside a desired wavelength window is removed from the light before the light reaches the exit.

39. A method of claim 33, wherein the fiber optic means comprises optical fibers having high core absorption and/or substantial light leakage into the cladding, at wavelengths outside of a desired wavelength window.

40. An method of claim 23, further comprises controlling the radiative power of the light delivered to the target by varying the electrical power input to the light source.

41. A method of claim 33, wherein the fiber optic means comprises at least two optical fibers, and the method further comprises coupling the optical fibers in a fiber coupling region, and controlling the radiative power of the light delivered to the target by movement of the optical fibers relative to maximum-performance positions of each optical fiber in the coupling region between the fibers emanating from the concentrator and the fibers that deliver light to the surgical target, by lateral or longitudinal misalignment.

42. A method of claim 33, further comprising a fiber coupling region and an iris located therein, wherein the fiber optic means comprises at least two optical fibers and radiative power of the light delivered to the target is controlled by the iris in the fiber coupling region.

43. A method of claim 23, wherein radiating high intensity incoherent light, the light source comprises a short-arc plasma discharge lamp.
